(19) United States
(12) Patent Application Publication
(10) Pub. No.: US 2022/0183952 A1
(43) Pub. Date: Jun. 16, 2022

(54) FORMULATION FOR THE TOPICAL TREATMENT OF THE SKIN AND RELATED USE

(71) Applicant: SVAS BIOSANA SPA, Somma Vesuviana (IT)

(72) Inventors: Umberto PERILLO, San Guiseppe Vesuviano (IT); Massimiliano PORZIO, Pozzuolia (IT)

(21) Appl. No.: 17/601,113

(22) PCT Filed: Jul. 29, 2019
(86) PCT No.: PCT/IB2019/056446

§ 371 Date: Oct. 4, 2021

(30) Foreign Application Priority Data
Jul. 27, 2018 (IT) 102018000007565

(51) Int. Cl. A61K 8/73; A61Q 19/00; A61K 8/63; A61K 8/9789; A61P 17/00

(52) U.S. Cl. CPC A61K 8/735; A61Q 19/00; A61P 17/00; A61K 8/9789; A61K 8/63

(57) ABSTRACT

A formulation for the topical treatment of the skin comprises an extract of Centella asiatica, a source of phytosterols with anti-oxidant and anty-inflammatory activity comprising Urtica dioica and hyaluronic acid or a salt thereof.

### TECHNICAL FIELD

The present invention finds application in the field of products and devices for the care or treatment of the body and particularly relates to a formulation for topical treatment of the skin, in particular for the preparation of creams, pomades, ointments, lotions, powders, medicated supports such as gauze, plasters and the like.

The invention also relates to a support for skin applications provided with such formulation and to the use of the formulation and the support.

### State of the Art

As known, one possible treatment of skin lesions such as pressure or vascular ulcers, bedsores and skin lesions of various etiology, is the localized topical treatment.

This type of treatment is carried out by localized application, at the lesion to be treated, of specific substances provided with one or more active principles conveyed by means of a base, such as a cream or an oil, possibly applied to a support such as a hydrophilic gauze or a plaster.

The tissue repair process is a very complex dynamic event, involving both resident and wound-infiltrating cells, responsible for the numerous biochemical and molecular processes.

The fundamental basis for the correct execution of the reparative process are provided through a series of phases, namely hemostasis-inflammation, proliferation, remodeling, and the coordinated action over time of a large number of cytokines, chemokines, growth factors, components of the extracellular matrix, proteases and corresponding cellular receptors.

In the inflammatory phase, platelets play a more complex role than simple hemostasis. They represent the reservoir of numerous molecules that they release upon their activation, including the growth factors PDGF, IGF1, EGF, FGF, TGF-β, which act by attracting inflammatory, endothelial, fibroblast and keratinocyte cells at the site of the lesion, thus determining the passage from the inflammatory phase to the proliferative one until reaching the remodeling of the lesion.

Moreover, in the proliferative phase of the tissue repair process it is important that the formation of the granulation tissue occurs.

In literature it is known the use of multiple substances, both of natural and synthetic origin, alone or in combination with other active ingredients.

For example, from the same Applicant, a composition is known for the topical localized treatment of the skin, also described in EP1909840, which includes Centella asiatica and Urtica dioica, in combination with further active ingredients and excipients.

In fact, the effectiveness of Centella asiatica is ascertained, and in particular of the relative triterpene fraction and its components, namely asiaticoside, Asian acid and madecassic acid, for the treatment of various skin lesions, including chronic ones.

Specifically, Centella asiatica operates in the proliferative phase favouring on one hand the synthesis of collagen, promoting the production of fibroblasts, and on the other hand favouring angiogenesis, promoting the synthesis of new blood vessels (neoangiogenesis).

In the same above cited patent, the Centella may be used in combination with a further vegetable source of triterpenes, in particular phytosterols, such as Urtica dioica, in order to obtain a synergistic effect thanks to the antioxidant and generally anti-aging effect of these active principles.

In particular, Urtica dioica also operates in the inflammatory phase, through an anti-inflammatory activity (COX 1 inhibition), antioxidant and vasodilator, mediated by the release of nitric oxide.

Further formulations for skin treatment with transdermal effect and including one of the above cited elements are known for example from US2011/0016213 and WO03/047609, whose compositions comprise Centella asiatica in extract form, US1994/0121027, wherein Centella asiatica is used in the form of an alcoholic extract, U.S. Pat. No. 6,113,926, which instead discloses a formulation for topical use comprising sources of sterols, such as Urtica dioica.

The formulation disclosed in the aforementioned EP1909840 has proved to be particularly effective but also suitable to be improved, in particular in order to associate further soothing and generally medical-type effects with the already known effects of Centella asiatica and Urtica dioica.

CN106580797 discloses a whitening cream that can also improve skin elasticity and which comprises phytosterols and sodium hyaluronate.

CN107184410 discloses an emollient cream which comprises among its ingredients phytosterols, sodium hyaluronate and Centella.

### Scope of the Invention

The object of the present invention is to overcome the above drawbacks by providing a formulation for topical treatment of the skin that has features of high efficiency and relative cheapness.

A particular object is to provide a formulation for topical treatment of the skin that has a high healing and soothing efficacy of skin lesions, including chronic ones.

Still another object is to provide a formulation for the topical treatment of the skin that combines the curative and soothing effect of the Centella with further effects that improve skin elasticity, increasing its local resistance at the injured areas and thus favouring the healing activity of Centella.

These objects, as well as others which will become more apparent hereinafter, are achieved by a formulation for topical treatment of the skin which, according to claim 1, comprises an extract of Centella asiatica, a source of phytosterols comprising Urtica dioica having a high antioxidant and anty-inflammatory power and hyaluronic acid or a salt thereof.

Thanks to this combination of elements the formulation will act even more effectively thanks to the synergistic effect of the three components.

As matter of fact, the action of hyaluronic acid will favour the formation of the granulation tissue so that the overall action of the selected principles favour the tissue repair process by acting on several levels.

Hyaluronic acid, an element forming the extracellular matrix, intervenes in the proliferative phase generating an optimal microenvironment capable of stimulating the increase of GFs, the proliferation and migration of fibroblasts, endothelial cells, keratinocytes and neoangiogenesis.

Advantageous embodiments of the invention are obtained according to the dependent claims.

### BEST MODES OF CARRYING OUT THE INVENTION

In its more general composition, a formulation for topical skin treatment essentially comprises an extract of Centella asiatica, a source of phytosterols and hyaluronic acid or a salt thereof.

Preferably, the Centella asiatica will be present in the form of a dry extract and in a percentage not exceeding 4% of the total weight of the formulation.

Even more preferably, the Centella asiatica extract is present in a percentage between 0.05% and 2% of the total weight of the formulation.

The use of Urtica dioica extract with antioxidant and anty-inflammatory activity is preferred, preferably dry extract, present in a percentage not exceeding 1% of the total weight, is preferred as source of plant phytosterols.

Even more preferably, the weight percentage of Urtica dioica will be between 0.005% and 0.05%.

Hyaluronic acid may be used in the form of salt and in particular of low molecular weight sodium hyaluronate (LMW—Low Molecular Weight).

Its weight percentage within the formulation will not exceed 1% of the total weight and will preferably be between 0.05% and 0.25%.

The formulation may be added with further dermatologically compatible active ingredients and excipients, such as antioxidants, preservatives, emulsifiers and humidifiers, as well as additional sources of phytosterols, such as carduus and nigella sativa.

In particular, in a preferred manner the use of powdered allantoin in a percentage not exceeding 1% of the total weight will be provided, so as to exploit the synergistic effect between allantoin and Centella asiatica extract which favours the re-epithelialization process, as already highlighted in EP1909840.

For the preparation of the formulation, the use of a mixture of polyethylene glycols (PEG) having a molecular weight between 200-4000 Da will also be preferred.

The following is a preferred but non-limiting example of the formulation according to the invention, wherein the various components are indicated according to weight percentages.

| | % |
|---|---|
| STEP A | |
| PEG 400 | 35.100 |
| PEG 1500 | 16.000 |
| PEG 4000 | 15.500 |
| Sorbitol | 7.300 |
| Vegetal glycerol | 4.500 |
| Water | 14.000 |
| STEP B | |
| Malaleuca Essntial Oil | 0.005 |
| Dry extract Centella asiatica | 0.100 |
| Edry extract Urtica Dioica | 0.015 |
| Menthol | 0.020 |
| Powdered Allantoin | 0.800 |
| LMW sodium hyaluronate | 0.200 |
| Water | 0.750 |
| STEP C | |
| Vaseline | 2.200 |
| Cetyl alcohol | 1.000 |
| stearyl alcohol | 1.000 |
| Phenoxyethanol | 1.000 |
| FASE D | |
| Tocopheryl acetate | 0.510 |

The formulation may be in the form of an ointment, cream, powder, lotion, solution or similar consistency.

The formulation may be applied directly to the skin at the lesion to be treated.

Alternatively, the formulation may be used to impregnate a gauze, in particular of a hydrophilic type, a plaster or other support suitable to be brought into contact with the skin for topical and localized treatment.

The use of Urtica dioica in the form of an extract, in particular dry extract, in association with hyaluronic acid or a salt thereof, for example sodium hyaluranate, has proved particularly effective, especially in its antioxidant action.

For the purposes of estimating the antioxidant potential of Urtica dioica (UE), the following table shows the comparison of the results of the FRAP (Ferric reducing antioxidant power) and TEAC (Trolox equivalent antioxidant capacity) assays of the UE with standard antioxidants, in particular hydroxytoluene butylate (BHT) and vitamin C. Both tests showed that the UE has a higher antioxidant capacity than the BHT.

| | | | Superoxide radical | Metal chelating |
|---|---|---|---|---|
| | FRAP | TEAC | scavenging activity, | ability, |
| | Mmol Fe2+/g | mmol TE/g | EC50, mg/ml | EC50, mg/ml |
| UE | 0.993 ± 0.018 | 1.5 ± 0.1 | 1.15 ± 0.02 | 241.6 ± 0.0 |
| Vitamin C | 6.57 ± 1.00 | 7.23 ± 0.28 | 0.06 ± 0.00 | N.D. |
| BHT | 0.416 ± 0.020 | 0.467 ± 0.005 | N.D. | N.D. |

The formulation and the support comprising the formulation according to the invention are susceptible of numerous modifications and variations, all of which are within the inventive concept expressed in the appended claims. All the details must be replaced by other technically equivalent elements, and the materials must be different according to requirements, without departing from the scope of protection of the present invention.

1. A formulation for the topical treatment of the skin, comprising:
an extract of Centella asiatica;
at least one a source of phytosterols with anti-oxidant and anty-inflammatory activity;
hyaluronic acid or a salt thereof;
characterized in that said at least one a source of phytosterols is an extract of Urtica dioica.

2. Formulation as claimed in claim 1, characterized in that said extract of Urtica dioica is present in a percentage not higher than 1% of the total weight.

3. Formulation as claimed in claim 2, characterized in that said extract of Urtica dioica is present in a percentage between 0.005% and 0.05% of the total weight.

4. Formulation as claimed in claim 3, characterized in that said extracts are dried extracts.

5. Formulation as claimed in claim 4, characterized in that said extracts of Centella asiatica is present in a percentage not higher than 4% of the total weight.

6. Formulation as claimed in claim 5, characterized in that said extract of Centella asiatica is present in a percentage ranging from 0.05% to 2% of the total weight.

7. Formulation as claimed in claim 1, characterized in that said hyaluronic acid salt is low molecular weight sodium hyaluronate.

8. Formulation as claimed in claim 7, characterized in that said sodium hyaluronate is present in a percentage not higher than 1% of the total weight and preferably between 0.05% and 0.25%.

9. Formulation as claimed in claim 7, characterized by comprising powdered allantoin in a percentage not higher than 1% of the total weight.

10. A support for the topical treatment of skin impregnated with a formulation comprising an extract of Centella asiatica, at least one source of phytosterols with anti-oxidant and anty-inflammatory properties and hyaluronic acid or a salt thereof,
characterized in that said at least one a source of phytosterols is an extract of Urtica dioica.

11. Use of a formulation comprising an extract of Centella asiatica, at least one source of phytosterols with anti-oxidant and anty-inflammatory properties and hyaluronic acid or a salt thereof for the topical treatment of the skin, wherein the formulation is applied in a localized manner on the skin either directly or by means of a support, such as a gauze or plaster, impregnated with said formulation,
characterized in that said at least one a source of phytosterols is an extract of Urtica dioica.
